(19) United States
(12) Patent Application Publication
(10) Pub. No.: US 2006/0136015 A1
(43) Pub. Date: Jun. 22, 2006

(54) Human body communication device, human body communication system and method using the same

(76) Inventors: Duck-Gun Park, Daejeon-city (KR); Sung-Weon Kang, Daejeon-city (KR); Jin-Kyung Kim, Daejeon-city (KR); Chang-Hee Hyoung, Daejeon-city (KR); Jin-Bong Sung, Daejeon-city (KR); Jung-Hwan Hwang, Daejeon-city (KR)

Correspondence Address: LADAS & PARRY LLP, 224 SOUTH MICHIGAN AVENUE, SUITE 1600, CHICAGO, IL, 60604, US

(21) Appl. No.: 11/284,749

(22) Filed: Nov. 22, 2005

(30) Foreign Application Priority Data
Dec. 8, 2004 (KR) 10-2004-0103031
Jun. 9, 2005 (KR) 10-2005-0049195

(51) Int. Cl. A61N 1/08

(52) U.S. Cl. 607/60

(57) ABSTRACT

A human body communication apparatus, a human body communication system, and a method using the same are provided. The communication apparatus is directly in contact with a human body and allows a small amount of current including a data signal to flow through the body so that information is communicated through the human body. In order to efficiently perform a data communication by preventing a collision of data packets in the human body communication system, one communication apparatus is selected from a plurality of communication apparatuses configuring the human body communication system to control or manage a communication order for other communication apparatuses and the respective apparatuses check whether the respective communication apparatuses transmit data through the human body to try a communication.

### CROSS-REFERENCE TO RELATED APPLICATION

This application claims priority to and the benefit of Korean Patent Applications 10-2004-0103031 and 10-2005-0049195 filed in the Korean Intellectual Property Office on Dec. 8, 2004 & May 6, 2005, the entire contents of which are incorporated herein by reference.

### BACKGROUND OF THE INVENTION

(a) Field of the Invention

The present invention relates to a human body communication apparatus, a human body communication system, and a method using the same.

(b) Description of the Related Art

Generally, human body communication is a communication method which transmits a signal in the form of a variation of electrical energy through a human body instead of a ‘wire’ of electrical products by using a principle that an electrical current flows through the human body.

Recently, various information communication apparatuses, such as a personal digital assistant (PDA), a portable personal computer, a digital camera, MP3 player, and a mobile phone, have been widely used. Users use information by transmitting/receiving e-mails and downloading data of contents through these apparatuses. These apparatuses use a physical communication line, such as a cable, as well as a predetermined adaptor or a connector in order that one communication device can transmit stored information to other communication apparatuses. Accordingly, data cannot be simply transmitted/received.

Also, when apparatuses for checking health status, for example, an electrocardiograph (ECG), a non-invasive blood pressure (NIBP), and a heart monitor, transmit the results to a computer or other communication apparatuses so that the results are used along with other information, a physical communication line must be used as the data are transmitted between the above-described communication apparatuses.

Particularly, when these apparatuses are connected by cables in the vicinity of the human body to perform the data communication, the cables apply a load on the human body and disturb mobility of the human.

The communication apparatuses and the apparatuses for checking heath status have common functions, such as display, memory, and digital signal processing functions, as well as unique functions for converting digital information into a voice signal or communicating with the Internet or other user. Accordingly, when these functions are properly applied, these apparatuses can efficiently use a communication resource and perform a simple and easy data communication. For this purpose, human body communication has been developed.

However, since these communication apparatuses can be used while moving to various sites of the human body and are frequently detached, it is necessary that new communication lines are rapidly formed through the human body according to the mobility of these apparatuses so as to enhance efficiency of human body communication.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a human body communication apparatus, a human body communication system and a method using the same in which data are transmitted through a human body as a medium having advantages of reducing power consumption and preserving a high communication performance.

An exemplary human body communication apparatus according to an embodiment of the present invention includes an electrode for transmitting/receiving a signal in the form of a current through the human body while being in contact with a human body, a preamplifier for amplifying the signal received through the electrode a band-pass filter for removing noise included in the signal amplified by the preamplifier, a comparator for comparing the filtered signal with the reference voltage, and a controller for recovering the signal output from the comparator and generating a base-band signal to be transmitted to the electrode.

An exemplary human body communication system according to an embodiment of the present invention includes a plurality of communication apparatuses for transmitting/receiving a base-band signal while being in contact with a human body, and a plurality of hosts having an operation performance and coupled with the respective communication apparatuses, wherein one communication apparatus (a master member) controls data transmission for a plurality of other communication apparatuses (slave members).

In a further embodiment, the human body communication system includes a plurality of communication apparatuses for transmitting/receiving a base-band signal while being in contact with a human body, and a plurality of hosts having an operation performance and coupled with the respective communication apparatuses, wherein the respective communication apparatuses broadcast an information request message to a plurality of other communication apparatuses when there is no signal transmitted through a human body.

An exemplary human body communication method for transmitting/receiving data in the form of a base-band signal through a human body wherein a plurality of communication apparatuses are in contact with the human body via an electrode and are respectively coupled with hosts configuring a network, according to an embodiment of the present invention, includes broadcasting an information request message from one communication apparatus (a master member) to other communication apparatuses (slave members) through the electrode, receiving a response message from the plurality of slave members that received the request message, configuring a member table of the plurality of slave members based on the received response message in a network, and broadcasting information of the member table.

In a further embodiment, an exemplary human body communication method for transmitting/receiving data in the form of a base-band signal through a human body wherein a plurality of communication apparatuses are in contact with the human body via an electrode and respectively coupled with hosts to configure a network includes monitoring whether the respective communication apparatuses perform data transmission through the human body, broadcasting an information notifying message from one communication apparatus to other communication apparatuses when the data transmission is in an idle state, receiving the response message from the other communication apparatuses that received the broadcast message, configuring a member table based on the received response message in a network, and determining a communication order for the other communication apparatuses based on the member table to start data communication.

### BRIEF DESCRIPTION OF THE DRAWINGS

FIG. 1 is a block diagram showing a communication apparatus according to an exemplary embodiment of the present invention.

FIG. 2 shows signal waveforms of the respective parts of a communication apparatus of FIG. 1.

FIG. 3 is a schematic view of a human body communication system according to an exemplary embodiment of the present invention.

FIG. 4 is an equivalent circuit of a human body communication system using a communication apparatus according to an exemplary embodiment of the present invention.

FIG. 5 is a flowchart for preparing a communication between a master member and a plurality of slave members in a human body communication system according to an exemplary embodiment of the present invention.

FIG. 6 is an exemplary member table composed by a master member in a process for preparing a communication of FIG. 5.

FIG. 7 is a flowchart for transmitting data from a master member to a slave member in a human body communication system according to an exemplary embodiment of the present invention.

FIG. 8 is a block diagram for configuring a data frame which a master member transmits to a slave member in FIG. 7.

FIG. 9 is a flowchart for receiving data with a slave member from a master member in a human body communication system according to an exemplary embodiment of the present invention.

FIG. 10 is a flowchart for transmitting data from a slave member to a master member in a human body communication system according to an exemplary embodiment of the present invention.

FIG. 11 is a flowchart showing a human body communication method according to another exemplary embodiment of the present invention.

### DETAILED DESCRIPTION OF THE EMBODIMENTS

Embodiments of the present invention will hereinafter be described in detail with reference to the accompanying drawings.

In the following detailed description, only certain exemplary embodiments of the present invention have been shown and described, simply by way of illustration. As those skilled in the art would realize, the described embodiments may be modified in various different ways, all without departing from the spirit or scope of the present invention.

Accordingly, the drawings and description are to be regarded as illustrative in nature and not restrictive. Like reference numerals designate like elements throughout the specification.

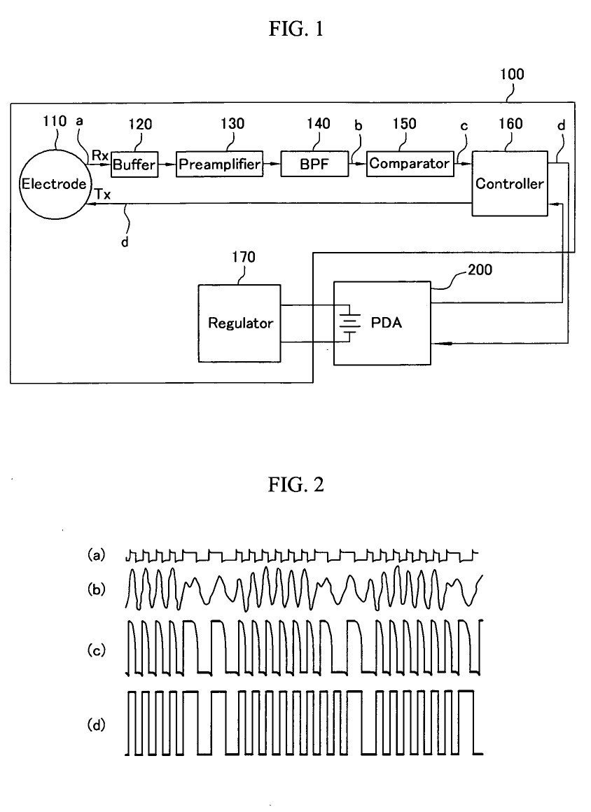

FIG. 1 is a block diagram showing a communication apparatus according to an exemplary embodiment of the present invention, and FIG. 2 shows signal waveforms of the respective parts of the communication apparatus of FIG. 1.

As shown in FIG. 1, according to an exemplary embodiment of the present invention, a communication apparatus 100 includes an electrode 110, a buffer 120, a preamplifier 130, a band-pass filter (BPF) 140, a comparator 150, a controller 160, and a regulator 170. The communication apparatus 100 is in contact with the human body to transmit information via the human body to a host (the PDA 200 shown in FIG. 1).

That is, the communication apparatus 100 is directly in contact with skin through the electrode 110 which flows a small amount of current into the skin to measure a body fat so that information is communicated through the human body.

Also, the communication apparatus 100 realizes the human body communication when it is coupled with the host 200 so that desired data are transmitted through the human body to other communication apparatuses and the other communication apparatuses are coupled with other hosts so that they receive, recover, and transmit a signal to the coupled hosts. In FIG. 1, the host 200 may use a personal digital assistant (PDA). However, the host may use other apparatuses having a self-operation function and data processing capability.

As shown in FIG. 1, the communication apparatus 100 does not include an RF module so that a base-band signal is transmitted via the current. The base-band signal is not frequency-modulated but pulse-code modulated (PCM).

The electrode 110 is directly in contact with the human body so that the current including the data flows through the human body. Also, the electrode 110 is directly in contact with the human body to receive the data signal. In FIG. 2, (a) shows a waveform of a signal transmitted from a part (a) of FIG. 1, that is, a signal transmitted from the electrode 110 of the communication apparatus 100.

The buffer 120 temporarily stores the received data to compensate for a difference of time or data transmission rate when data are transmitted to the function blocks in the communication apparatus 100. In this case, the communication apparatus 100 can have reduced interference between the transmitted signals and efficiently consume the current by increasing an input impedance of the buffer 120. The interference is generated by a self-impedance of the human body used as a communication channel.

The preamplifier 130 amplifies the signal transmitted through the buffer 120 to help a recovery of the signal.

The band-pass filter 140 eliminates noise included in the received signal during a transmission process. The noise is generated due to component heterogeneity of the human body used as a transmission channel, and is added to the signal during the transmission process and eliminated by the band-pass filter 140. In FIG. 2, (b) shows a waveform of a signal transmitted from a part (b) of FIG. 1, that is, a signal amplified and noise-eliminated through the buffer 120, the preamplifier 130, and the band-pass filter 140.

The comparator 150 compares the noise-eliminated signal with the reference voltage to output the signal in the form of a binary code signal thereby preventing an error of the data transmission. In FIG. 2, (c) shows a waveform of a signal transmitted from a part (c) of FIG. 1, that is, a signal generated through the comparator 150.

The controller 160 detects an error of the data signal input through the comparator 150 to be corrected and recovers the signal received through the electrode 110. In this case, the controller 160 analyzes the received data frame and recovers a clock and data. In order to recover the clock, it must know start and end timing points of the respective codes. Such information may be included in a preamble of the data frame to be transmitted. The controller 160 analyzes the preamble as soon as it receives data to recognize the recovery start and end timing points, correct a period thereof, and detect the error. The recovered signal may be transmitted to the host 200 coupled with the communication apparatus 100 to be stored in a memory of the host 200 (not shown).

The controller 160 quantizes, encodes, and pulse-code modulates data received from the host 200 to generate a base-band signal. The generated base-band signal is transmitted through the electrode 110 to the human body. In this case, the controller 160 selects an encoding scheme appropriate for a communication status to generate a signal. The generated data stream may have various types of signals such as a nonreturn-to-zero (NRZ) signal, a return-to-zero (RZ) signal, and a Manchestor signal. In FIG. 2, (d) shows a waveform of a signal transmitted from a part (d) of FIG. 1, that is, a signal recovered and generated through the controller 160.

The controller 160 defines a communication order and rule between the communication apparatuses during the data communication process to prevent data collision phenomenon and to increase transmission efficiency of the data to prevent waste of communication resources.

The regulator 170 provides an offset voltage between the communication apparatus 100 and the host 200.

According to an exemplary embodiment of the present invention, the communication apparatus 100 pulse-code modulates the analogue signal to be converted into the base-band digital signal. Since the signal is not frequency-modulated by an RF module, loss of power becomes small.

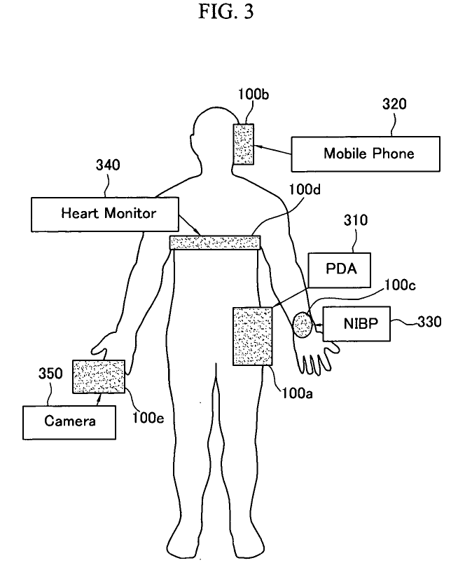

FIG. 3 is a schematic view of a human body communication system according to an exemplary embodiment of the present invention. As shown in FIG. 3, a plurality of hosts 200, for example, communication apparatuses, such as a PDA 310, a mobile phone 320, and a digital camera 350, and various apparatuses for measuring health status, such as an NIBP 330 and a heart monitor 340, may be used in the vicinity of the human body. These hosts 200 are respectively coupled with a plurality of communication apparatuses 100a to 100e to realize the human body communication system. Also, the respective communication apparatuses 100a to 100e have electrodes directly in contact with the human body to realize the human body communication system.

The communication apparatuses 100 configuring the human body communication system as shown in FIG. 3 generate a base-band signal to transmit the same through the human body. Accordingly, a low pass channel is formed in the human body to perform a half-duplex communication. The low pass channel has a bandwidth wide enough to accommodate frequencies of a series of data in the human body. Thus, when the data are transmitted from both sides of the communication channel, which is configured by contacting the plurality of communication apparatuses 100 with the human body, data collision generates a loss. Accordingly, these communication apparatuses 100 must be controlled by the communication rule and order.

According to an exemplary embodiment of the present invention, in order to efficiently perform data communication by preventing a collision of data packets in the human body communication system, a master/slave method is used such that one communication apparatus is selected from a plurality of communication apparatuses configuring the human body communication system to control and manage a communication order for other communication apparatuses and a listen before speak method checks whether the respective communication apparatuses transmit data through the human body to try a communication. These two methods will be described in detail.

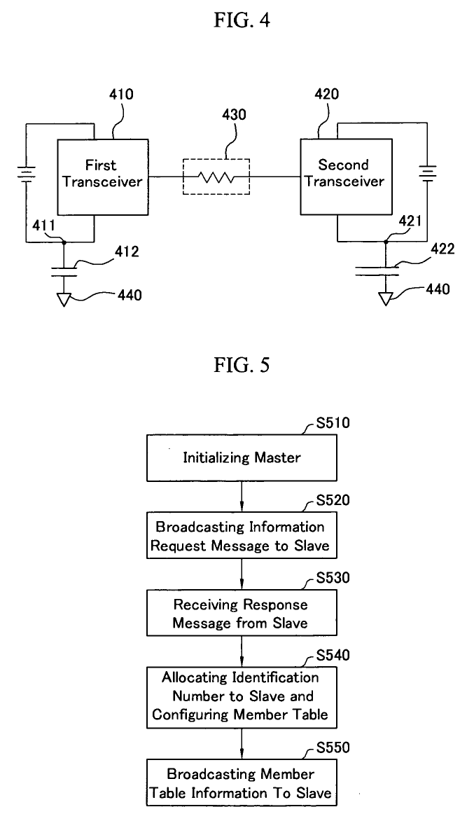

FIG. 4 is an equivalent circuit of a human body communication system using a communication apparatus according to an exemplary embodiment of the present invention. As shown in FIG. 4, the human body may be represented as a resistance 430, and a first communication apparatus 410 and a second communication apparatus 420 transmit/receive a current via the resistance 430 to perform a data communication. In this case, since a ground voltage of the communication apparatuses 410 and 420 is different from an earth ground voltage 440, small capacitors 412 and 422 may be provided between the ground voltage of the communication apparatuses 410 and 420 and the earth ground voltage. The function of these capacitors 412 and 422 is performed by the regulator 170 in FIG. 1.

FIG. 5 is a flowchart for preparing a data communication between one communication apparatus and a plurality of other communication apparatuses in a human body communication system according to an exemplary embodiment of the present invention, which relates to a half-duplex communication type of a master/slave communication method.

The one communication apparatus (hereinafter called ‘master member’ while the others are called ‘slave members’ for better comprehension and ease of description) is selected among a plurality of communication apparatuses used in a human body communication system as shown in FIG. 3 so as to manage the entire communication system.

Any communication apparatus may be selected as a master member in the system as long as the communication apparatus has enough operation speed and internal memory to efficiently process data considering an operation capability of the coupled host. Since the master member and all slave members are the same communication apparatuses 100, the respective data transmission/reception capabilities are the same. The slave members may be selected to perform the function of the master member as long as one master member is used in the embodiment. For example, in the human body communication system as shown in FIG. 3, the communication apparatus 100a coupled with the PDA 310 can be used as a master member.

In a master/slave communication scheme, the master member controls the respective communications for the slave members such that the master member relays a data communication between the plurality of slave members.

When one communication apparatus is selected as a master member among the plurality of communication apparatuses used in a human body communication system and power is applied to the communication apparatuses 100 coupled with the master member, an internal operation is reset and initialized. By the reset, an initial environment is established, variables are initialized, and a plurality of slave members are recognized at the controller 160 of the master member to prepare a data communication which transmits data through the human body as a medium (S510). In this case, the controller 160 can select an appropriate form of a pulse code modulated signal to be encoded.

The master member broadcasts an information request message, such as the respective types of the hosts, to the slave members so as to obtain the information in the human body communication system (S520).

When the slave members receive the broadcast message and transmit a response message including the slave member information with a random interval of time, the master member receives the response messages during a predetermined time to recognize the existence of the slave members (S530). In this case, since transmission orders of the response message and time slots are not allocated between the plurality of slave members, the respective slave members can repeatedly and periodically transmit the response message so as to cope with a transmission error due to collision of the transmitted messages.

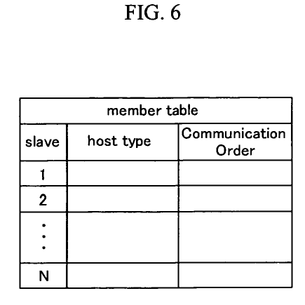

The controller 160 allocates unique identifiers, such as an identification number and a node ID, for the corresponding slave members and configures a member table for managing the slave members to be stored (S540). The master member manages all data communications for the slave members included in the member table. Also, the master member determines a communication order for the slave members using the information of the slave members included in the member table such that the master member is communicated with the specified one slave member. FIG. 6 is an exemplary member table composed by a master member in a process for preparing a communication of FIG. 5. As shown in FIG. 6, the member table may include the ID numbers of the slave members, types of the hosts coupled with the slave members, and the communication order.

The controller 160 broadcasts the member table so that the respective slave members recognize other members in the system (S550). The master member may repeatedly and periodically transmit the information so as to cope with a slave member not receiving the message including the member table information due to data collision. In this case, the slave members need not transmit an ACK message after receiving the member table. Also, the slave member can request the master member to relay a data communication between other slave members included in the received member table.

The hosts may not be fixed, but may be often added or removed according to determination of the user. Accordingly, in order to actively cope with the variation and thereby efficiently perform a human body communication, the master member can periodically broadcast an information request message to the slave members and repeat the processes shown in FIG. 5 to update the member table.

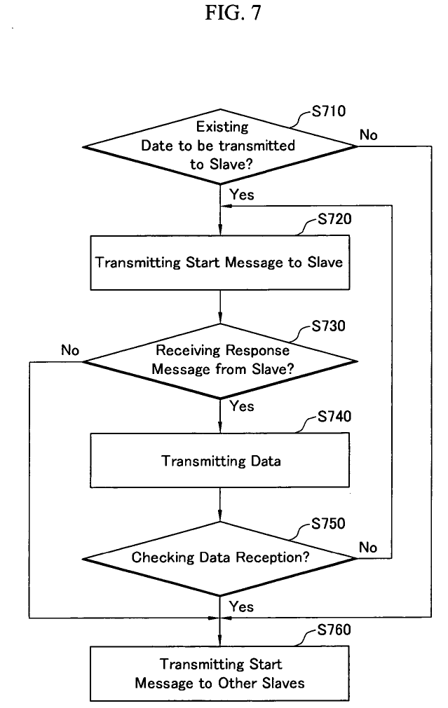

FIG. 7 is a flowchart for transmitting data from a master member to a slave member in a human body communication system according to an exemplary embodiment of the present invention.

The controller 160 determines a communication order of the slave members in the system and performs a communication according to the communication order. The master member determines whether there are data to be transmitted to the slave member corresponding to the communication order (S710). When there is no data to be transmitted, the master member prepares a communication for the next slave member (S760).

When there are data, the master member transmits a data transmission start message to the corresponding slave member so that the slave member prepares a data communication with the master member (S720).

The slave member receives the start message and transmits a response message to the master member so that the master member transmits desired data to the slave member (S730, S740). When the master member does not receive a response message from the slave member, the corresponding slave member is removed from the communication order and the next slave member is prepared to be communicated with (S730, S760).

When the slave member receives the data from the master member, the slave member transmits an ACK message to the master member. When the master member receives the ACK message, the start message is transmitted to the next slave member so that the above processes are repeated to perform a communication (S750, S760).

When the master member does not receive the ACK message within a predetermined time after it transmits the data to the specified slave member, the master member retransmits the start message to the corresponding slave member to try a communication again (S750,S720).

As described above, since data transmission is performed only if the master receives the response message from the slave member, the communication order may be omitted in the member table configuring process of the master member or added, that is, it may be excluded in the member table. The excluded slave members are not controlled by the master member. Thus, an information transmission error due to a collision of data packets can be prevented between these excluded communication apparatuses. Also, when the slave members are removed from the system after configuring the member table or the slave members (not to be communicated with due to problems of the communication apparatuses 100 or hosts 200) are deleted from the communication order, the resource of the master member can be efficiently used and the components can be controlled at a real-time in a communication network.

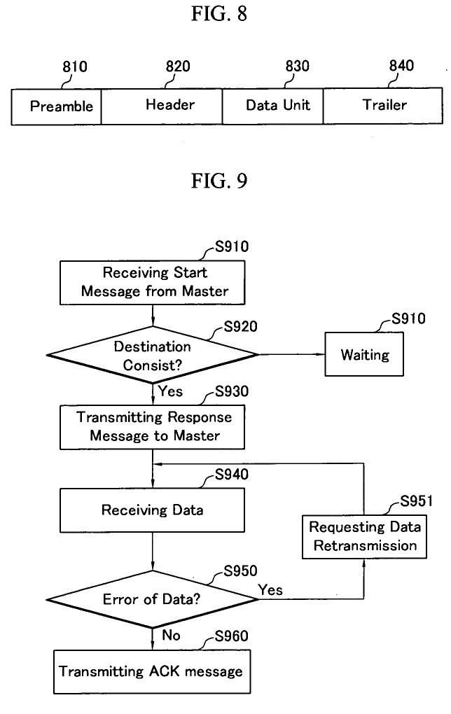

FIG. 8 is a block diagram for configuring a data frame which a master member transmits to a slave member in FIG. 7. As shown in FIG. 8, a data frame includes a preamble 810, a header 820, a data unit 830, and a trailer 840.

The preamble 810 is information for exactly recovering data received by the slave members so as to synchronize a transmission timing point between the master member and the slave members.

The header 820 includes identification information of the slave members for receiving the corresponding data frame and notifies a destination of the frame.

The data unit 830 is data which the master member desires to transmit to the slave member. The trailer 840 is an end part of the data frame and may include information for detecting errors.

In this embodiment, a data frame is transmitted from the master member to the slave member as shown in FIG. 8. However, it is obvious that similar data frames are transmitted from the slave members to the master member.

FIG. 9 is a flowchart for a slave member receiving data from a master member in a human body communication system according to an exemplary embodiment of the present invention.

A disordered slave member waits until it receives a data transmission start message from the master member (S910). The disordered slave member does not correspond to the communication order.

The slave member receives the start message, analyzes the header 820 and recognizes whether a destination corresponds to the slave member. When the destination is consistent with the corresponding slave member, the slave member transmits a response message to the master member and waits for a data transmission of the master member (S920, S930).

When the destination is not consistent with the corresponding slave member, the slave member waits without transmitting any response messages, such as a NAK message (S920, S921).

The slave member analyzes the received data frame and determines whether there is an error. When there is no error at a data transmission, the slave member transmits an ACK message to the master member (S950, S960). When there is an error at a data transmission, the slave member requests the master member to retransmit the corresponding data (S950, S951).

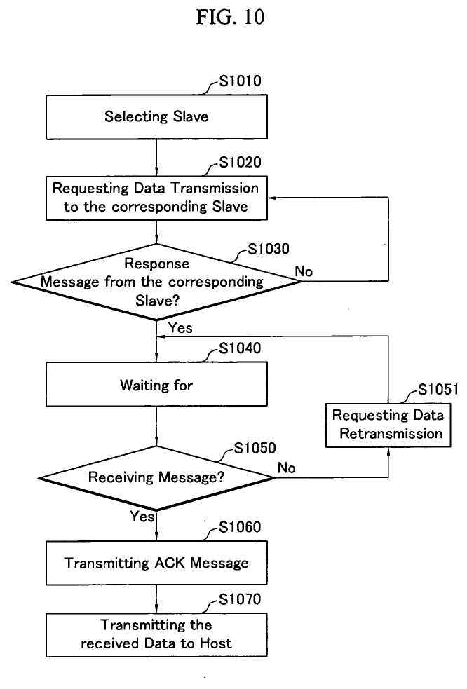

FIG. 10 is a flowchart for transmitting data from a slave member to a master member in a human body communication system according to an exemplary embodiment of the present invention. FIG. 10 shows a data transmission that is performed from the slave member to the master member in a reverse direction of that of FIG. 8 and FIG. 9.

When the master member configures the member table with the slave members, the communication order and backward communication orders are determined for the respective slave members.

First, the controller 160 of the master member checks the member table content to select the slave member corresponding to the communication order and checks whether there is a data to be transmitted by the slave member (S1010, S1020).

When the master member receives a response message for notifying a data existence from the corresponding slave member, the master member requests a data transmission start to the slave member and waits (S1030, S1040). When the master member does not receives the response message from the slave member, the master member may request retransmission of data to the corresponding slave member (S1030, S1020), or may try a communication for the next slave member while excluding the corresponding slave member in the communication order.

When the master member receives the response message from the slave member but does not receive the data within a predetermined time, the master member requests data retransmission to the corresponding slave member (S1050, 1051). When the data is not successfully transmitted even after the data retransmission requests are repeated, the next slave member may be tried to perform a communication.

When the data is transmitted within a predetermined time, the ACK message is transmitted to the corresponding slave member (S1060). When the slave member receives the message, the slave member waits until it is corresponding to the next communication order or receives a broadcast message for requesting information.

The master member processes the received data at the controller 160 to store the processed data at the memory of the communication apparatus 100 or transmits the same to the host 200 coupled with the master member (S1070). Also, the master member can retransmit the received data to other slave members when requested. The host 200 can transmit the received data to communication terminals, such as a computer, or a mobile phone, of an external area of the human body communication system.

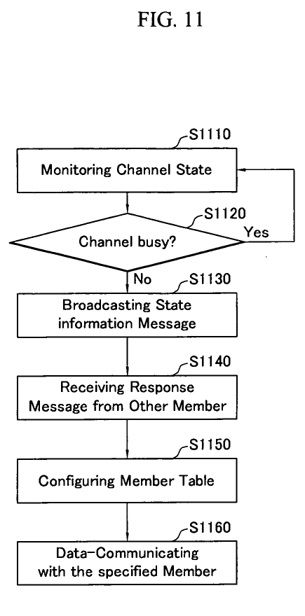

FIG. 11 is a flowchart showing a human body communication method according to another exemplary embodiment of the present invention. FIG. 11 shows a human body communication method using a listen before speak scheme wherein instead of one communication apparatus (e.g., a master member) being established so as to control a communication order between all communication apparatuses, the respective communication apparatuses consecutively monitor whether data are directly transmitted through a human body and try a communication when data are not transmitted through a human body, that is, when the communication channel is idle.

As shown in FIG. 3, the communication apparatuses 100a to 100e are coupled with the plurality of hosts and are directly in contact with the human body to monitor whether data are consecutively transmitted through the human body (S1110). In this case, the controller 160 of the respective communication apparatuses 100 can use an encoding method in which clock information is incorporated in the data stream, for example, a Manchestor coding scheme.

The Manchestor coding scheme uses two types of pulses, that is, a negative pulse and a positive pulse, so as to express one code, unlike the data stream of the NRZ scheme or RZ scheme data stream. Accordingly, since the two pulses have a half width of the code, when the polarity of the pulse is converted during 0.5×(1/data transfer rate)(sec), a data communication state in which data are transmitted through the human body, that is, a busy state, can be recognized.

When the respective communication apparatuses consecutively monitor a data transmission performed in the vicinity of the human body to recognize an idle state (S1110, S1120), the respective communication apparatuses broadcast an information notifying message to other communication apparatuses (S1130). The information may include identification information and concerning information, such as types of hosts.

The other communication apparatuses successfully receive a message broadcast from the specified communication apparatus without a loss of data due to collision of data packets and transmit an ACK message to the broadcast communication apparatus. The controller 160 of the communication apparatus receives such a response message so that it configures a member table with other communication apparatuses of the same communication network and stores the member table at the internal memory (S1140, S1150).

The respective members consecutively monitor a channel status based on the member table configured in this manner and perform a data communication for other members (S1160). As in the master/slave scheme, when the specified communication apparatus determines that the channel is idle as a result of monitoring, an information request message is repeatedly and periodically broadcast. In this case, when the message is not transmitted from other communication apparatuses within a predetermined time, the member table can be updated by deleting the corresponding slave master from the given member table.

As described above, according to an exemplary embodiment of the present invention, since the respective communication apparatuses construct the respective communication area without the specified element for controlling the entire network, a plurality of communication networks can be overlapped on the basis of the specified one communication apparatus in the same human body communication system.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

According to an exemplary embodiment of the present invention, since a human body can be used as a channel of data transmission so that data are not leaked due to hacking, a half-duplex data transmission can be performed.

Since data are transmitted using a base-band signal without using an RF module, energy can be efficiently used.

Also, variables of various communication apparatuses used in the vicinity of the human body can be applied in a human body communication system at real-time.

What is claimed is:

1. A human body communication apparatus comprising:
an electrode for transmitting/receiving a signal in the form of a current through a human body while being in contact with the human body;
a preamplifier for amplifying the signal received through the electrode;
a band-pass filter for removing noise included in the signal amplified by the preamplifier;
a comparator for comparing the filtered signal with a reference voltage; and
a controller for restoring the signal output from the comparator and generating a base-band signal to be transmitted to the electrode, wherein the controller generates the base-band signal by a pulse coding modulation.

2. A human body communication system comprising:
a plurality of communication apparatuses for transmitting/receiving a base-band signal while being in contact with a human body; and
a plurality of hosts having an operation performance, and coupled with the respective communication apparatuses,
wherein one communication apparatus (a master member) controls a data transmission for a plurality of other communication apparatuses (slave members).

3. The human body communication system of claim 2, wherein the communication apparatus comprises:
an electrode for transmitting/receiving a signal in the form of a current through a human body while being in contact with the human body;
a preamplifier for amplifying the signal received through the electrode;
a band-pass filter for removing noise included in the amplified signal;
a comparator for comparing the filtered signal with the reference voltage; and
a controller for recovering the signal output from the comparator and generating a base-band signal to be transmitted to the electrode.

4. The human body communication system of claim 3, wherein
the master member is established based on an operation performance of the respective hosts coupled with the plurality of communication apparatuses.

5. The human body communication system of claim 3, wherein the master member obtains information of a plurality of slave members, configures a member table of the slave members based on the obtained information, and determines a communication order of the slave members.

6. The human body communication system of claim 5, wherein the master member broadcasts the member table to the slave members included in the member table.

7. The human body communication system of claim 2, wherein the master member relays a data transmission from the corresponding slave member to other slave members according to a request of one slave member.

8. A human body communication system comprising:
a plurality of communication apparatuses for transmitting/receiving a base-band signal while being in contact with a human body; and
a plurality of hosts having an operation performance and coupledwith the respective communication apparatuses,
wherein the respective communication apparatuses broadcast an information request message to a plurality of other communication apparatuses when there is no signal transmitted through the human body.

9. The human body communication system of claim 8, wherein the respective communication apparatuses configure a member table based on information of the other communication apparatuses that transmitted a response message on the broadcast message, and tries a data transmission while consecutively monitoring whether data are transmitted through the human body.

10. A human body communication method for transmitting/receiving data in the form of a base-band signal through a human body wherein a plurality of communication apparatuses are in contact with the human body via an electrode and are respectively coupled with hosts configuring a network, the human body communication method comprising:
broadcasting an information request message from one communication apparatus (a master member) to other communication apparatuses (slave members) through the electrode;
receiving a response message from the slave members that received the request message;
configuring a member table of the slave members based on the received response messages in a network; and
broadcasting information of the member table.

11. The human body communication method of claim 10, wherein on configuring the member table, the master member determines a communication order for the slave members and the member table includes the determined communication order.

12. The human body communication method of claim 11, further comprising periodically broadcasting the information request message to update the member table in the network.

13. The human body communication method of claim 11, further comprising:
transmitting a communication start message to the slave member corresponding to the communication order;
receiving a response message for the communication start message from the slave member;
transmitting data from the master member to the slave member; and
detecting an error of the data transmission and transmitting an ACK message to the master member when there is no error.

14. The human body communication method of claim 13, wherein the master member stops a communication for the corresponding slave member and transmits a communication start message to the next slave member, when the master member does not receive the response message for the communication start message from the slave member.

15. The human body communication method of claim 13, wherein the slave member analyzes a header part of the received data to determine whether a destination is consistent with the corresponding slave member and waits without transmitting any response messages until it is consistent with the same.

16. The human body communication method of claim 11, wherein the master member transmits the data received from the corresponding slave member to the host when the slave member corresponding to the communication order has data to be transmitted to the master member.

17. The human body communication method of claim 16 wherein the host transmits the received data to a communication terminal of an external area of the human body network.

18. The human body communication method of claim 16 wherein the master member retransmits the data to other slave members when the slave member requests for the master member to transmit the transmitted data to other slave member.

19. A human body communication method for transmitting/receiving data in the form of a base-band signal through a human body, wherein a plurality of communication apparatuses are in contact with the human body via an electrode and are respectively coupled with hosts to configure a network, the method comprising:
monitoring whether the respective communication apparatuses perform a data transmission through the human body;
broadcasting an communication apparatus information notifying message from the one communication apparatus to other communication apparatuses when the data transmission is in an idle state;
receiving the response message from the other communication apparatuses that received the broadcast message;
configuring a member table based on the received response messages in a network; and
determining a communication order for the other communication apparatuses based on the member table to start a data communication.

20. The human body communication method of claim 19, further comprising periodically broadcasting the communication apparatus information notifying message from the communication apparatus configuring the member table and updating the member table based on a response message for the broadcast message.

21. The human body communication method of claim 19, wherein the communication apparatus uses an encoding method such as the Manchestor coding scheme.
